(19) United States
(12) Patent Application Publication
(10) Pub. No.: US 2011/0313465 A1
(43) Pub. Date: Dec. 22, 2011

(54) METHOD AND APPARATUS FOR SPINAL STABILIZATION

(75) Inventors: Christopher Warren, Aliso Viejo, CA (US); Robert Flower, Sun City, CA (US); Fausto Olmos, Laguna Niguel, CA (US); Walter Cuevas, Irvine, CA (US)

(73) Assignee: INTERVENTIONAL SPINE, INC., Irvine, CA (US)

(21) Appl. No.: 13/162,329

(22) Filed: Jun. 16, 2011

Related U.S. Application Data
(60) Provisional application No. 61/355,418, filed on Jun. 16, 2010.

(51) Int. Cl. A61B 17/88; A61B 17/70

(52) U.S. Cl. 606/279; 606/246

(57) ABSTRACT

A method and apparatus of limiting at least one degree of movement between a superior vertebrae and an inferior vertebrae of a patient includes advancing a distal end of a stabilization device made of a bio-absorbable material, such as cortical bone, into a pedicle of the inferior vertebrae. A proximal portion of the stabilization device is positioned such that the proximal portion limits at least one degree of movement between a superior vertebrae and an inferior vertebrae by contacting a surface of the superior vertebrae.

### CROSS-REFERENCE TO RELATED APPLICATIONS

The present application claims priority under 35 U.S.C. §119(e) to U.S. Provisional Application No. 61/355,418 filed on Jun. 16, 2010, the disclosure of which is incorporated by reference herein in its entirety.

### BACKGROUND

1. Field

The present invention relates to medical devices and, more particularly, to methods and apparatuses for spinal stabilization.

2. Description of the Related Art

The human spine is a flexible weight bearing column formed from a plurality of bones called vertebrae. There are thirty three vertebrae, which can be grouped into one of five regions (cervical, thoracic, lumbar, sacral, and coccygeal). Moving down the spine, there are generally seven cervical vertebra, twelve thoracic vertebra, five lumbar vertebra, five sacral vertebra, and four coccygeal vertebra. The vertebra of the cervical, thoracic, and lumbar regions of the spine are typically separate throughout the life of an individual. In contrast, the vertebra of the sacral and coccygeal regions in an adult are fused to form two bones, the five sacral vertebra which form the sacrum and the four coccygeal vertebra which form the coccyx.

In general, each vertebra contains an anterior, solid segment or body and a posterior segment or arch. The arch is generally formed of two pedicles and two laminae, supporting seven processes—four articular, two transverse, and one spinous. There are exceptions to these general characteristics of a vertebra. For example, the first cervical vertebra (atlas vertebra) has neither a body nor spinous process. In addition, the second cervical vertebra (axis vertebra) has an odontoid process, which is a strong, prominent process, shaped like a tooth, rising perpendicularly from the upper surface of the body of the axis vertebra. Further details regarding the construction of the spine may be found in such common references as Gray's Anatomy, Crown Publishers, Inc., 1977, pp. 33-54, which is herein incorporated by reference.

The human vertebrae and associated connective elements are subjected to a variety of diseases and conditions which cause pain and disability. Among these diseases and conditions are spondylosis, spondylolisthesis, vertebral instability, spinal stenosis and degenerated, herniated, or degenerated and herniated intervertebral discs. Additionally, the vertebrae and associated connective elements are subject to injuries, including fractures and torn ligaments and surgical manipulations, including laminectomies.

The pain and disability related to the diseases and conditions often result from the displacement of all or part of a vertebra from the remainder of the vertebral column. Over the past two decades, a variety of methods have been developed to restore the displaced vertebra to their normal position and to fix them within the vertebral column. Spinal fusion is one such method. In spinal fusion, one or more of the vertebra of the spine are united together (“fused”) so that motion no longer occurs between them. The vertebra may be united with various types of fixation systems. These fixation systems may include a variety of longitudinal elements such as rods or plates that span two or more vertebrae and are affixed to the vertebrae by various fixation elements such as wires, staples, and screws (often inserted through the pedicles of the vertebrae). These systems may be affixed to either the posterior or the anterior side of the spine. In other applications, one or more bone screws may be inserted through adjacent vertebrae to provide stabilization.

Although spinal fusion is a highly documented and proven form of treatment in many patients, there is currently a great interest in surgical techniques that provide stabilization of the spine while allowing for some degree of movement. In this manner, the natural motion of the spine can be preserved, especially for those patients with mild or moderate disc conditions. In certain types of these techniques, flexible materials are used as fixation rods to stabilize the spine while permitting a limited degree of movement.

Notwithstanding the variety of efforts in the prior art described above, these techniques are associated with a variety of disadvantages. In particular, these techniques typically involve an open surgical procedure, which results higher cost, lengthy in-patient hospital stays and the pain associated with open procedures.

Therefore, there remains a need for improved techniques and systems for stabilization the spine. Preferably, the devices are implantable through a minimally invasive procedure.

### SUMMARY

Accordingly, some embodiments a spinal stabilization device comprises an elongate body having a distal end and a proximal end. The distal end can be implanted in an inferior vertebrae and the proximal end can abut against a superior vertebrae to limit at least one degree of movement between the superior vertebrae and the inferior vertebrae. In some embodiments, the body can at least partially be made of an allograft, such as cortical bone. In some embodiments, the body can be at least partially made of a biocompatible material, such as Polyether-etherketone (PEEK™) and can be an interbody cage.

In some embodiments, the spinal stabilization can comprise a proximal anchor, carried by the elongate body, and having a diameter. In some embodiments, the spinal stabilization device can further comprising a distal anchor on the distal end of the elongate body.

Some embodiments of the present application comprise a method of limiting extension between an inferior and superior body structure of a spine. The steps of the method can include inserting a distal end of a biocompatible stabilization device into the inferior body structure of the spine and securing the stabilization device to the inferior body structure. In some embodiments, at least a portion of the stabilization device can be an abutment that limits extension between the superior body structure and the inferior body structure. In some embodiments, cortical bone can grow around the proximal end of the stabilization device to form the abutment.

### BRIEF DESCRIPTION OF THE DRAWINGS

FIG. 1A a side elevational view of a portion of a vertebra having some embodiments of a stabilization device implanted therein.

FIG. 1B is a posterior view of a portion of a vertebra having two devices similar to that of FIG. 1A implanted substantially bilaterally therein.

FIG. 2 is a cross-sectional view of some embodiments of a stabilization device.

FIG. 3 is a side perspective view of some embodiments of a stabilization device having a proximal anchor.

FIG. 4 is a side elevational view of the stabilization device of FIG. 3 with a partial cross-sectional view of the proximal anchor.

FIG. 5 is a side elevational view of some embodiments of a stabilization device having a distal anchor.

FIG. 6A is an exploded side perspective view of some embodiments of a stabilization device.

FIG. 6B is a side elevational view of the stabilization device of FIG. 6A with the proximal anchor attached to the body.

FIG. 7A is a side perspective view of some embodiments of a stabilization device with the proximal anchor attached to the body.

FIG. 7B is an exploded side perspective view of the stabilization device of FIG. 7A.

FIG. 8 is a side elevational view of a body of FIG. 7A.

FIG. 9 is a side elevational view of a proximal anchor section of FIG. 7A.

FIG. 10 is a side perspective view of some embodiments of a stabilization device with the proximal anchor attached to the body.

FIG. 11 is a cross-sectional view of a stabilization device of FIG. 10.

FIG. 12 is a posterior elevational view of a portion of a vertebra with portions thereof removed to receive a fixation device.

FIG. 13 is a posterior elevational view of a portion of a vertebra having two devices similar to that of FIG. 1A implanted substantially bilaterally therein and a member extending between the two devices.

### DETAILED DESCRIPTION OF THE PREFERRED EMBODIMENTS

Although the stabilization devices described herein will be disclosed primarily in the context of a spinal stabilization procedure, the methods and structures disclosed herein are intended for application in any of a variety medical applications, as will be apparent to those of skill in the art in view of the disclosure herein.

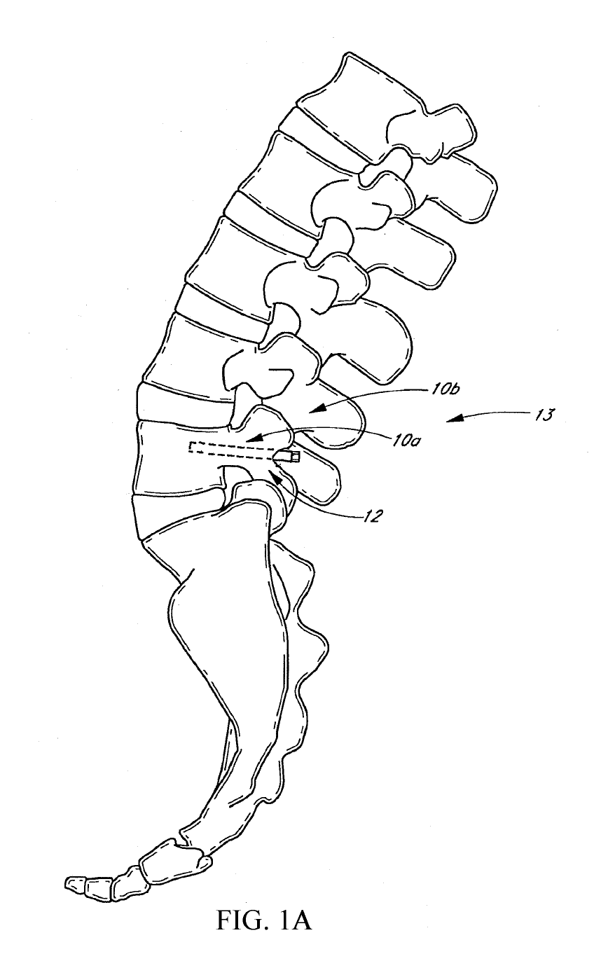

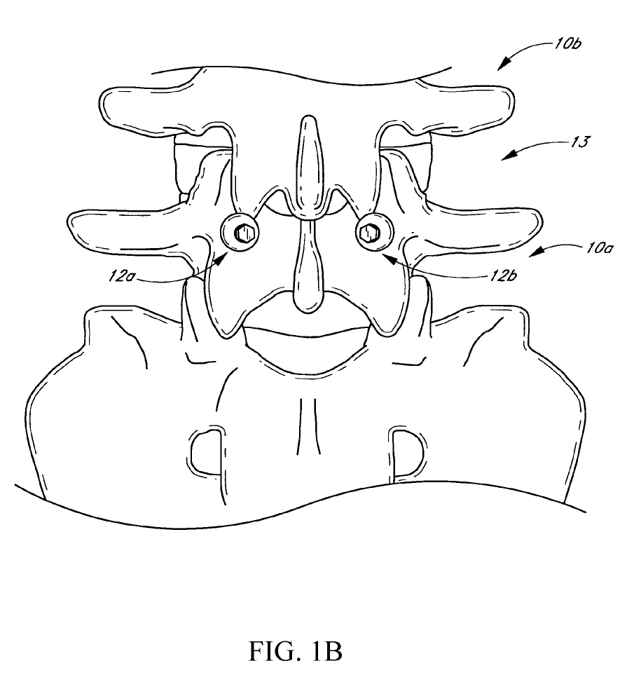

FIGS. 1A and 1B are side and rear elevational views of a pair of bone stabilization devices 12, positioned within a body structure 10a of the spine. As will be explained in detail below, the bone stabilization device 12 may be used in a variety of techniques to stabilize the spine. In some embodiments, the device 12 is attached (e.g., inserted or screwed into) and/or coupled to a single body structure and limits motion of a second body structure. In some embodiments, the device 12 limits extension in the spine by being attached and/or coupled to an inferior body structure and limiting motion of an adjacent superior body structure. “Body structure” as used herein is the anterior solid segment and the posterior segment of any vertebrae of the five regions (cervical, thoracic, lumbar, sacral, and coccygeal) of the spine. In some embodiments, the device limits motion by contacting, abutting against and/or wedging against the adjacent body structure and/or a device coupled to the adjacent body structure.

With reference to the illustrated embodiment of FIGS. 1A and 1B, the distal end of the bone stabilization device 12 is inserted into the pedicle of the inferior vertebrae, preferably through the pars (i.e., the region between the lamina between and the superior articular processes). The proximal end of the device 12 extends above the pars such that it limits motion of a superior adjacent vertebrae 10b with respect to the inferior adjacent vertebrae 10b. In some embodiments, the proximal end of the device limits motion by abutting and/or wedging against a surface of the superior adjacent vertebrae as the superior adjacent vertebrae moves relative to the inferior adjacent vertebrae. In this manner, at least one degree of motion between the inferior and superior vertebrae may be limited. For example, the spine generally has six (6) degrees of motion which include flexion, extension, left and right lateral bending and axial rotation or torsion. In the illustrated embodiment, at least extension of the spine is limited. Embodiments in which the devices are inserted with bilateral symmetry can be used to limit left and right lateral bending.

In the illustrated embodiment, motion of the spine is limited when the proximal end of the device contacts, abuts, and/or wedges against the inferior articular process of the superior adjacent vertebra 10b. In this application, it should be appreciated that one or more intermediate member(s) (e.g., plates, platforms, coatings, cement, and/or adhesives) can be coupled to the superior adjacent vertebra 10b or other portions of the spine that the device contacts, abuts, and/or wedges against. Thus, in this application, when reference is made to the device contacting, abutting and/or wedging against a portion of the spine it should be appreciated that this includes embodiments in which the device contacts, abuts and/or wedges against one or more intermediate members that are coupled to the spine unless otherwise noted.

As explained below, the bone stabilization devices 12 may be used after laminectomy, discectomy, artificial disc replacement, microdiscectomy, laminotomy and other applications for providing temporary or permanent stability in the spinal column. For example, lateral or central spinal stenosis may be treated with the bone fixation devices 12 and techniques described below. In such procedures, the bone fixation devices 12 and techniques may be used alone or in combination with laminectomy, discectomy, artificial disc replacement, and/or other applications for relieving pain and/or providing stability.

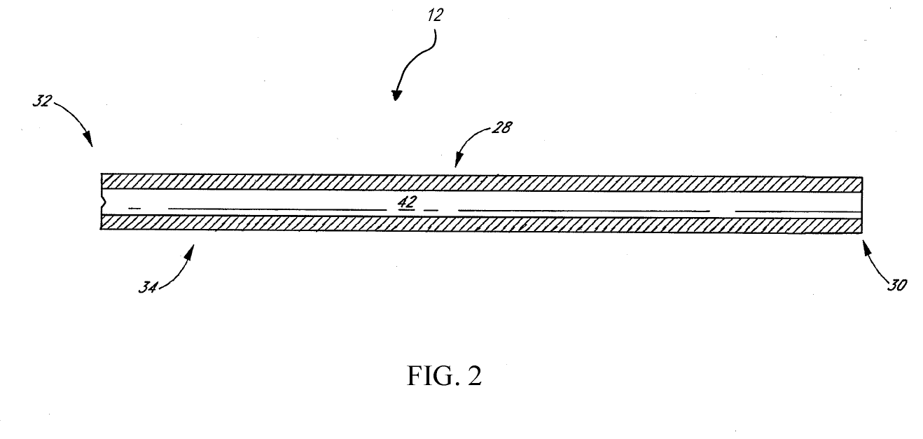

An embodiment of the stabilization device 12 will now be described in detail with initial reference to FIGS. 2-4. The stabilization device 12 can be a dowel-like device comprising a body 28 that extends between a proximal end 30 and a distal end 32, as illustrated in FIG. 2. The length, diameter and construction materials of the body 28 can be varied, depending upon the intended clinical application. In embodiments optimized for spinal stabilization in an adult human population, the body 28 will generally be within the range of from about 20-90 mm in length and within the range of from about 3.0-8.5 mm in maximum diameter. Of course, it is understood that these dimensions are illustrative and that they may be varied as required for a particular patient or procedure. As discussed below, when the stabilization device 12 is implanted in the patient, the proximal end 30 can extend beyond the surface of the pedicle of the inferior vertebrae and abut against the inferior facet of the superior adjacent vertebrae. In this manner, motion between the adjacent vertebrae may be limited and/or constrained.

In some embodiments, the body 28 can be made of allograft or other suitable biological material, such as for example cortical bone, cancellous bone, demineralized bone matrix (DBM) or bone morphogenic protein (BMP). The biological material can beneficially promote integration of the stabilization device 12 into surrounding tissue. However, as will be described in more detail below, other materials, or bioabsorbable or biocompatible materials can be utilized, depending upon the dimensions and desired structural integrity of the finished stabilization device 12.

In certain embodiments, the body 28, can be made entirely of allograft bone (e.g., cortical bone, cancellous bone and/or a combination of cortical and cancellous bone allograft). However, as note above other materials, or bioabsorbable or biocompatible materials can be utilized, depending upon the dimensions and desired features in other embodiments. For example, in one embodiment, the body 28 is substantially made entirely of allograft bone such that over 95% of the weight of the body 28 is from allograft bone, in another embodiment, over 90% of the weight of the body 28 is from allograft bone and in another embodiment over 75% of the weight of the body 28 is from allograft bone. In some embodiments, the body 28 can be formed of allograft bone and certain portions can be formed or coated with another biocompatible or bioabsorbable material, such as, a metal (e.g., titanium), ceramics, nylon, Teflon, polymers, etc. In other embodiments, a portion of the body 28 is formed from allograft bone while the remaining portions are made of another material metal (e.g., titanium), ceramics, nylon, Teflon, polymers. For example, portions of the body 28 that are intended to contact the spine can be formed of allograft bone with the remaining portions formed of another material (e.g., metal, ceramic, nylon, polymer etc.).

In certain embodiments, the body 28 can be press-fitted (i.e., have an interference fit) with the hole in the inferior vertebrae 10a. As such, the diameter of the body 28 is preferably the same as, or slightly larger than, the diameter of the hole formed in the vertebrae. In some embodiments, once the stabilization device 12 is inserted into the hole formed in the inferior vertebrae 10a, the stabilization device 12 can fuse with the inferior vertebrae 10a. The allograft or biocompatible material promotes bone growth around the stabilization device 12.

In other embodiments, the body 28 can be attached to the vertebrae through the use of an adhesive, such as biomedical cement. In some embodiments, a temporary adhesive can be used to initially secure the stabilization device 12 to the vertebrae until the components can fuse together.

In still other embodiments, the body can have a distal end that is configured to be secured to the hole in the inferior vertebrae 10a. As explained in further detail below, the distal end can have a bone anchor that secures the body to the inferior vertebrae. For example, the distal end can have threads to screw the body to the inferior vertebrae. In another example, the body can have groove features around the circumference of the body that help grab the bone after inserting the body into the hole after, for example the anchor is press-fitted into the hole. In some embodiments, other methods of securing the stabilization device to the vertebrae can be used, such as for example flanges, fasteners, staples, screws, and the like.

The proximal end 30 of the stabilization device 12 can extend beyond the surface of a inferior vertebrae 10a such that it can limit motion of an adjacent superior vertebrae 10b with respect to the inferior vertebrae 10a. In some embodiments, the proximal end 30 of the device can limit motion by abutting and/or wedging against a surface of the superior vertebrae 10b as the superior vertebrae 10b moves relative to the inferior vertebrae 10a. In some embodiments, a mass or growth can form around the proximal end 30 of the stabilization device 12, which can provide a support against which the superior vertebrae 10b can abut. The mass can advantageously grow around the superior vertebrae 10a and provide an abutment that is contoured to the shape of the superior vertebrae 10a. In some embodiments, at least a portion of the proximal end 30 of the stabilization device 12 can have a roughened surface to promote bone growth around the proximal end 30. In some embodiments, the proximal end 30 can have a structure or a cage to serve as a foundation for bone growth. In some embodiments, the proximal end 30 can have other features that are known to promote bone growth.

With continued reference to FIG. 2, in some embodiments, the body 28 can be cannulated forming a central lumen 42 to accommodate installation over a placement wire as is understood in the art. The cross section of the illustrated central lumen is circular but in other embodiments may be non-circular, e.g., hexagonal, to accommodate a corresponding male tool for installation or removal of the body 28 as explained below. In other embodiments, the body 28 may partially or wholly solid.

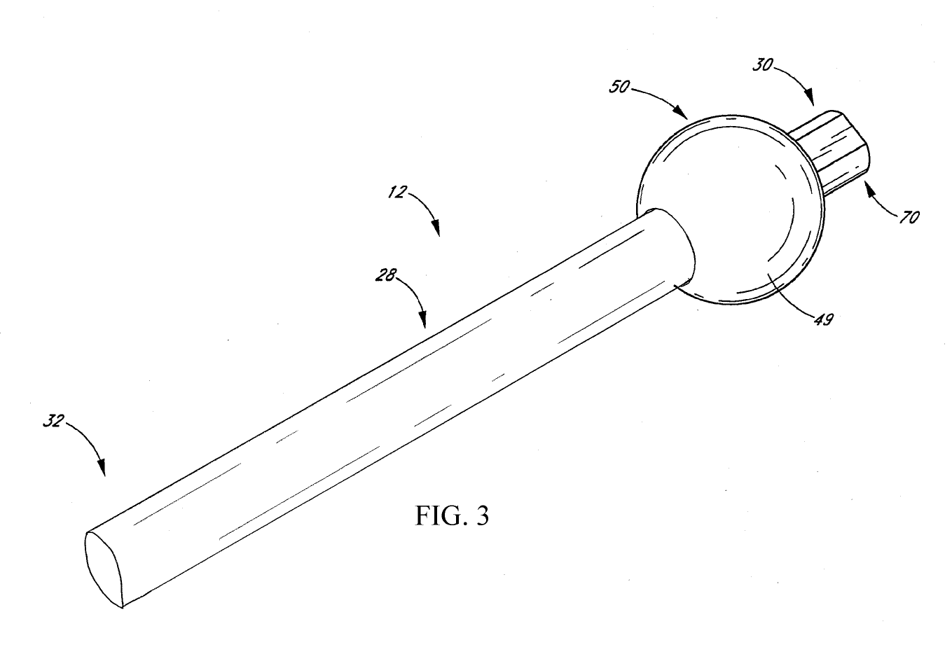

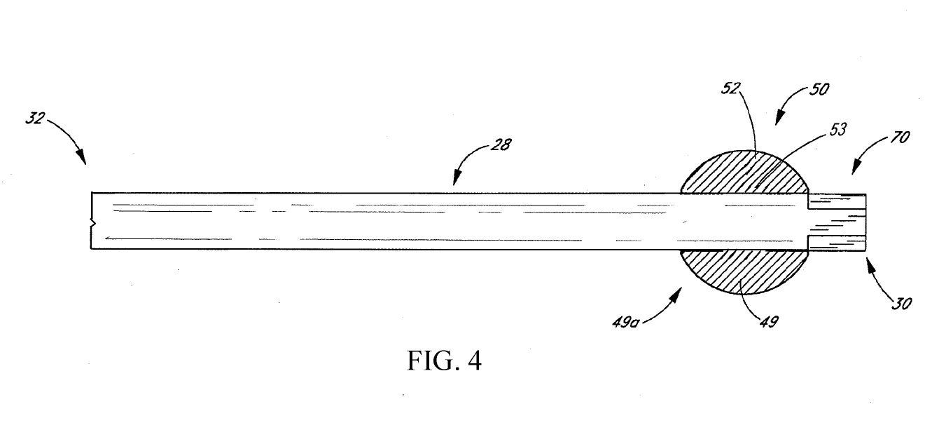

As illustrated in FIGS. 3 and 4, in some embodiments, the proximal end 30 of the fixation device can be provided with a proximal anchor 50. As illustrated in FIG. 4, the proximal anchor 50 can comprise a housing 52, which forms a lumen 53 configured such that the body 28 can extend, at least partially, through the proximal anchor 50.

In the embodiment illustrated in FIGS. 3 and 4, the outer surface 49 of the proximal anchor 50 has a smooth or spherical shape. As will be explained below, the outer surface 49 of the proximal anchor 50 can be configured to abut against the inferior facet of the superior adjacent vertebrae. In this manner, motion between the adjacent vertebrae may be limited and/or constrained.

In embodiments optimized for spinal stabilization in an adult human population, the anchor 50 can have a diameter within the range of from about 1 to 1/16 of an inch in some embodiments the proximal anchor proximal anchor 50 within the range from about 0.5 to ⅛ of an inch in some embodiments.

In some embodiments, the proximal anchor 50 of the fixation device can be coupled to, attached, or integrally formed with the body 28. For example, the proximal anchor 50 can be preassembled with the body 28 prior to implanting the device. The proximal anchor 50 can be attached to the body 28 through an interference fit, or press fit. In other embodiments, the proximal anchor 50 can be attached to the body 28 with the use of adhesives, fasteners, staples, screws, and the like. In another example, the proximal anchor 50 and the body 28 can be made from a single piece. Thus, the clinician can select a single-piece fixation device of the proper length, or preassemble a body 28 and proximal anchor 50 of desired dimensions, and advance the device into the vertebrae until the proximal anchor lies flush with the vertebrae or is otherwise positioned accordingly with respect to the vertebrae.

In some embodiments, the body 28 and proximal anchor 50 can be implanted as separate components, wherein the proximal anchor 50 is attached to the body 28 in situ. The clinician can have several stabilization devices 12 with an array of bodies 28 and proximal anchors 50, having, for example, different configurations and/or shapes. The clinician can choose the appropriate body 28 and secure the body to the inferior vertebrae 10a according to any method known in the art, such as the methods discussed above. Then, the clinician can assess the position of the body 28 with respect to the superior vertebrae and chose the proximal anchor 50 from the array, which best fits the patient anatomy to achieve the desired clinical result. The proximal anchor 50 can be advanced onto body 28 until the proximal anchor 50 lies flush with the vertebrae or is otherwise positioned accordingly with respect to the vertebrae. In some embodiments, the proximal anchor 50 can advantageously be coupled to body 28 after the body 28 is partially or fully inserted into the vertebrae. The proximal anchor 50 can be secured to the body 28 through an interference fit, adhesives, fasteners, staples, screws, and the like.

In some embodiments, the proximal anchor 50 can be made entirely of allograft bone (e.g., cortical bone, cancellous bone, demineralized bone matrix (DBM) or bone morphogenic protein (BMP and/or a combination or subcombinatnion of such elements). However, as note above other materials, or bioabsorbable or biocompatible materials can be utilized, depending upon the dimensions and desired features in other embodiments. For example, in one embodiment, the proximal anchor 50 is substantially made entirely of allograft bone such that over 95% of the weight of the proximal anchor 50 is from allograft bone, in another embodiment, over 90% of the weight of the proximal anchor 50 is from allograft bone and in another embodiment over 75% of the weight of the proximal anchor 50 is from allograft bone. In some embodiments, the proximal anchor 50 can be formed of allograft bone and certain portions can be formed or coated with another biocompatible or bioabsorbable material, such as, a metal (e.g., titanium), ceramics, nylon, Teflon, polymers, etc. In other embodiments, a portion of the proximal anchor 50 is formed from allograft bone while the remaining portions are made of another material metal (e.g., titanium), ceramics, nylon, Teflon, polymers. For example, portions of the proximal anchor 50 that are intended to contact the spine can be formed of allograft bone with the remaining portions formed of another material (e.g., metal, ceramic, nylon, polymer etc.).

In some embodiments, the proximal anchor that is coupled to, attached or integrally formed with the body 28 can be configured to have an outer surface which can rotate, preferably freely, with respect to the body 28. This arrangement advantageously reduces the tendency of the body 28 to rotate and/or move within the inferior vertebrae as the proximal anchor 50 contacts the superior vertebrae.

With reference to FIGS. 3-5, in some embodiments the proximal end 30 of the body 28 can have a rotational coupling 70, for allowing the body 28 to be rotated. Rotation of the rotational coupling 70 can be utilized to rotationally drive the distal anchor 34 into the bone. In such embodiments, any of a variety of rotation devices can be utilized, such as electric drills or hand tools, which allow the clinician to manually rotate the proximal end 30 of the body 28. Thus, the rotational coupling 70 can have any of a variety of cross sectional configurations, such as one or more curved faces, flats or splines. In the illustrated embodiment, the rotational coupling 70 is a male element in the form of a hexagonal projection. However, in other embodiments, the rotational coupling 70 can be in the form of a female component, machined, milled or attached to the proximal end 30 of the body 28. For example, in some embodiments, the rotational coupling 70 can comprise an axial recess with a polygonal cross section, such as a hexagonal cross section. In some embodiments, the axial recess may be provided as part of the central lumen 42.

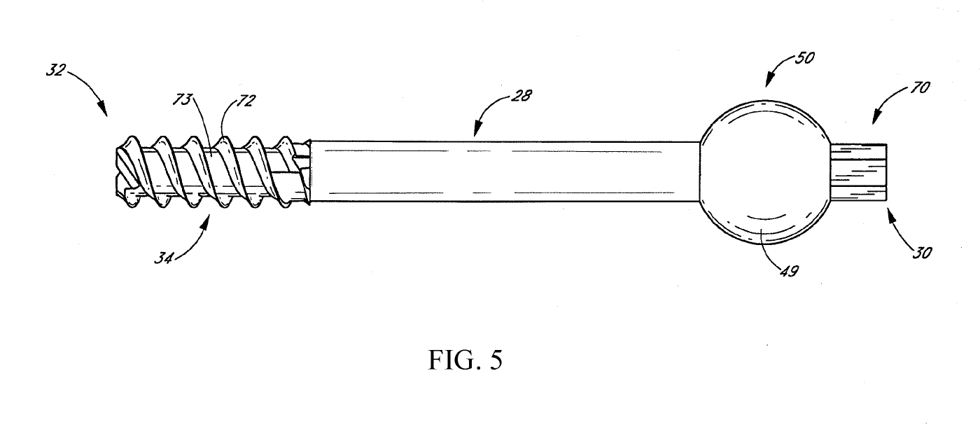

As discussed above, in some embodiments, the body 28 has a dowel-like shape and can be press-fitted into a hole in the inferior vertebrae 10a. However, in other embodiments, the distal end 32 of the body 28 can be provided with a cancellous bone anchor and/or distal cortical bone anchor in the form of a thread. Generally, for spinal stabilization, the distal anchor 34 can be adapted to be rotationally inserted into a portion (e.g., the pars or pedicle) of a first vertebra. In the embodiment illustrated in FIG. 5, the distal anchor 34 comprises a helical locking structure 72 for engaging cancellous and/or distal cortical bone. In the illustrated embodiment, the locking structure 72 comprises a flange that is wrapped around a central core 73, which can be generally cylindrical in shape. The flange 72 can extend through at least one and generally from about 2 to about 50 or more full revolutions depending upon the axial length of the distal anchor 34 and intended application. The flange can generally complete from about 2 to about 60 revolutions. The helical flange 72 is preferably provided with a pitch and an axial spacing to optimize the retention force within cancellous bone. While the helical locking structure 72 is generally preferred for the distal anchor, it should be appreciated that other distal anchors can comprise other structures configured to secure the device in the cancellous bone anchor and/or distal cortical bone. In some embodiments, the length of the helical anchor can range from about 8 mm to about 80 mm.

In some embodiments, the helical flange 72 can have a generally triangular cross-sectional shape. However, it should be appreciated that the helical flange 72 can have any of a variety of cross sectional shapes, such as rectangular, oval or other as deemed desirable for a particular application through routine experimentation in view of the disclosure herein. For example, in one modified embodiment, the flange 72 can have a triangular cross-sectional shape with a blunted or square apex. One particularly advantageous cross-sectional shape of the flange are the blunted or square type shapes. Such shapes can reduce cutting into the bone as the proximal end of the device is activated, reducing a “window-wiper effect” that is caused by cyclic loading and can loosen the device 12. The outer edge of the helical flange 72 can define an outer boundary. The ratio of the diameter of the outer boundary to the diameter of the central core 73 can be optimized with respect to the desired retention force within the cancellous bone and giving due consideration to the structural integrity and strength of the distal anchor 34. Another aspect of the distal anchor 34 that can be optimized is the shape of the outer boundary and the central core 73, which in the illustrated embodiment are generally cylindrical.

The distal end 32 and/or the outer edges of the helical flange 72 may be atraumatic (e.g., blunt or soft). This inhibits the tendency of the stabilization device 12 to migrate anatomically distally and potentially out of the vertebrae after implantation. Distal migration can also be inhibited by the dimensions and presence of the proximal anchor 50. In the spinal column, distal migration can be particularly disadvantageous because the distal anchor 34 may harm the tissue, nerves, blood vessels and/or spinal cord which lie within and/or surround the spine. Such features also reduce the tendency of the distal anchor to cut into the bone during the “window-wiper effect.” In other embodiments, the distal end 32 and/or the outer edges of the helical flange 72 can be sharp and/or configured such that the distal anchor 34 is self tapping and/or self drilling.

The fixation devices 12 can be made of allograft or other suitable biological material, such as for example cortical bone. The biological material can beneficially promote integration of the stabilization device into surrounding tissue.

In one embodiment, the distal end of the fixation device 12 that is configured to be positioned within the inferior vertebral body is intended to integrate with the implantation site. In contrast, the proximal or stabilizer portion of the device which abuts against the superior vertebral body can be treated with an extra process such that this portion of the device resists bone in growth and integration. For example, the proximal or stabilizer portion that abuts against the superior vertebra body can be sterilized (e.g., with gamma radiation, ebeam or Ethylene Oxide (ETO)) and/or treated through a chemical process that causes such portions to resist bone in growth and integration. In a similar manner, the distal end of the fixation device can also be treated to enhance bone in growth and integration.

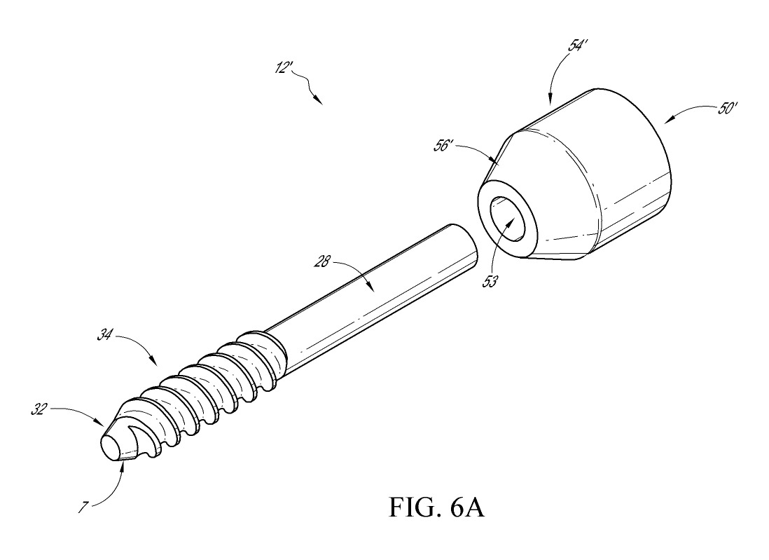

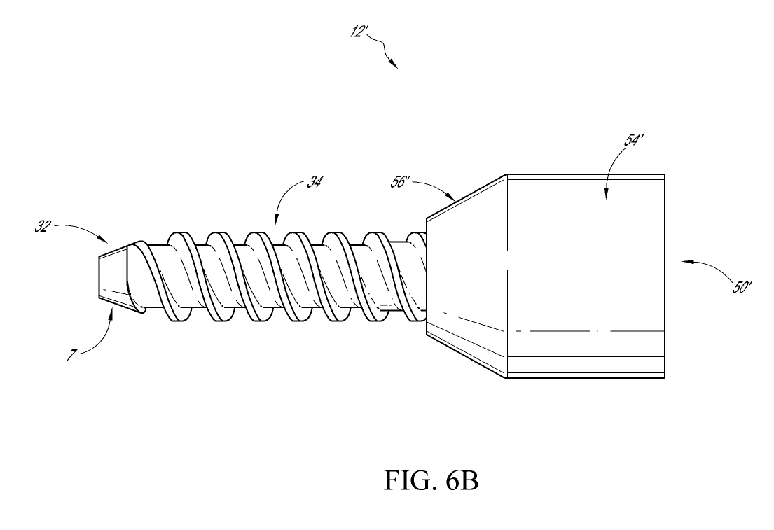

FIGS. 6A and 6B illustrate an embodiment of device 12′ with a proximal anchor 50′ and distal anchor 34. The illustrated proximal anchor 50′ has a cylindrical proximal portion 54′ and a tapered distal portion 56′. The cylindrical proximal portion 54′ is configured to abut the inferior facet of the superior vertebrae. The tapered distal portion 56′ is configured to fit in the countersinks 300 on the inferior vertebrae. As illustrated in FIG. 6A, the proximal anchor 50′ can have a lumen 53′ configured to couple with the body 28. In some embodiments, the lumen 53′ can be coupled to the body 28 in a variety of manners, such as, adhesives, cements, fasteners, threaded surfaces, interlocking surface structures and the like. In the illustrated embodiment, the proximal anchor 50′ is press-fitted onto the body 28.

In the illustrated embodiment of FIGS. 6A and 6B, the distal end 32 is provided with atraumatic or blunt tip 7. This feature can reduce the tendency of the distal anchor to cut into the bone during the “window-wiper effect” that is caused by cyclic loading of the device as described above.

In the illustrated embodiment embodiments, the body 28 and proximal anchor 50′ can be made from allograft. As discussed above, the allograft material advantageously promotes integration with the native bone structure. In some embodiments, it can be beneficial for the proximal anchor 50′ not to integrate with the native bone structures, such as when the proximal anchor 50′ needs to be removed later, or when bone growth around the proximal anchor 50′ undesirably changes critical dimensions of the proximal anchor. In such embodiments, the proximal anchor 50 can be treated with a process to resist bone in-growth and integration.

In some embodiments, the proximal anchor 50 can be configured such that it can be removed after being coupled and advance over the body 28. In this manner, if the clinician determines after advancing the proximal anchor that the proximal anchor 50 is not of the right or most appropriate configuration (e.g., size and/or shape), the clinician can remove the proximal anchor 50 and advance a different proximal anchor 50 over the body 28. In such an embodiment, the proximal anchor 50 is preferably provided with one or more engagement structures (e.g., slots, hexes, recesses, protrusions, etc.) configured to engage a rotational and/or gripping device (e.g., slots, hexes, recesses, protrusions, etc.). Thus, in some embodiments, the proximal anchor 50 can be pulled and/or rotated such that the anchor 50 is removed from the body 28.

In one embodiment of the device of FIGS. 6A and 6B, the body 28 has a length of about 25-30 millimeters and a diameter of about 4.5 millimeters. The proximal anchor 50′ can have a length of about 11 millimeters and a diameter of about 10 millimeters.

As noted above, on one embodiment, the proximal anchor 50, 50′ described above (and/or the proximal end of the device of FIG. 2) can be treated with a process to resist bone in growth and integration into this portion of the device.

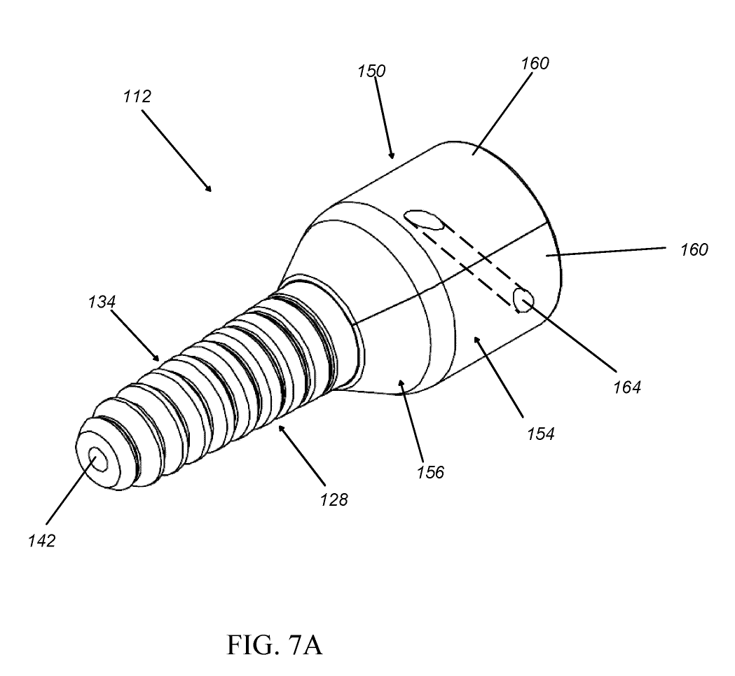

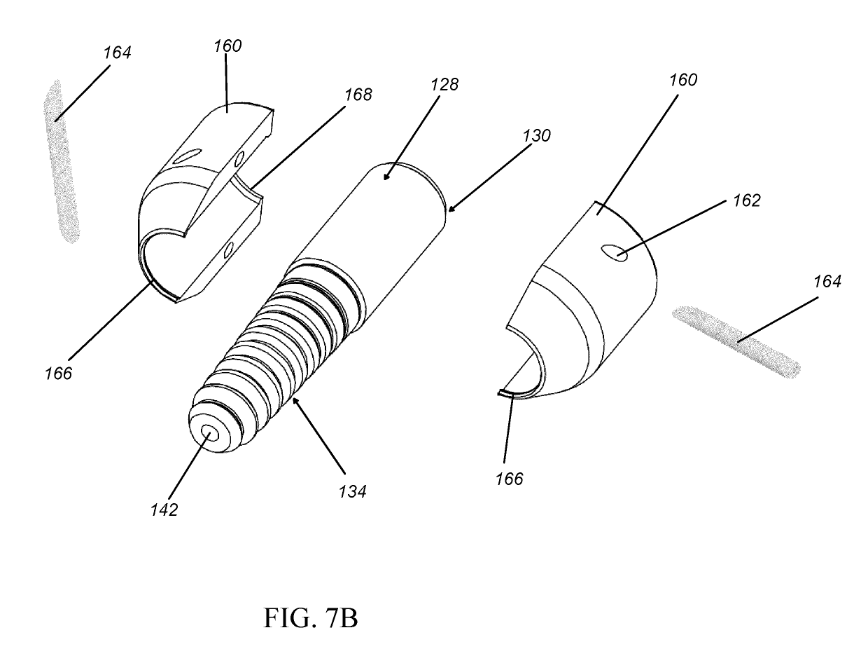

FIGS. 7A and 7B illustrate another embodiment of a device 112 having a proximal anchor 150 and a distal anchor 134. In the illustrated embodiment, the proximal anchor 150 has a cylindrical proximal portion 154 and a tapered distal portion 156. The cylindrical proximal portion 154 is configured to abut the inferior facet of the superior vertebrae and the tapered distal portion 156 is configured to fit in the countersinks 300 on the inferior vertebrae (and/or to extend above the vertebrae surface if a counter sink is not used). As illustrated in FIG. 7B, the proximal anchor 150 can be made of two proximal anchor sections 160 that are coupled together. In the illustrated embodiments, each of the two proximal anchor sections 160 is the same and makes up half of the proximal anchor 150. The two proximal anchor sections 160 are configured to couple to each other. In other embodiments, the two proximal anchor sections can have different designs that are configured to attach to each other.

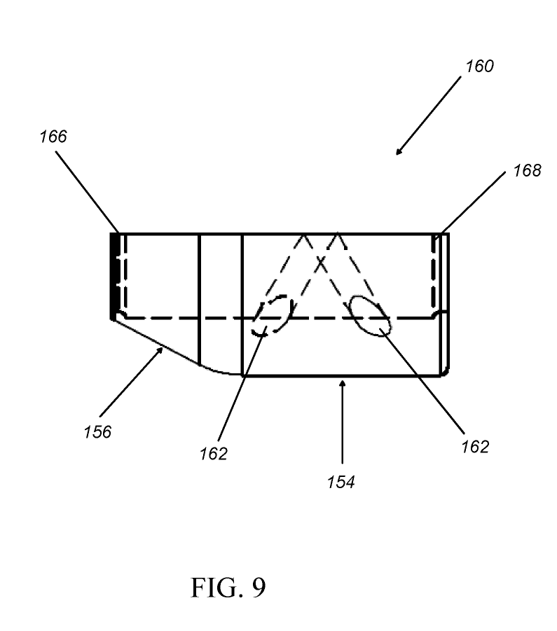

With continued reference to FIGS. 7A and 7B, the proximal anchor sections 160 can be secured around the body 128 towards the proximal end 130. Once the proximal anchor sections 160 are positioned around the body 128, they can be secured to each other with a pin or dowel 164. The dowel 164 can be inserted into holes 162 in the proximal anchor sections 160 that extend from one proximal anchor section 160 to the other proximal anchor section 160, as illustrated in FIG. 7A. In some embodiments, the hole 162 can extend at an angle to the transverse plane that is normal to the longitudinal axis of the proximal anchor 150, as best illustrated in FIG. 9. The dowel 164 can have an interference fit in the holes 162 or can be secured through a variety of manners, such as, adhesives, cements, fasteners, threaded surfaces, interlocking surface structures and the like. For example, instead of a dowel, a fastener can be screwed through one of the proximal anchor sections 160 and into the other proximal anchor section 160 to secure the two sections together. In one arrangement, the pin or dowel 164 is also made of allograft. In a modified arrangement, the pin or dowel can be made of another material (e.g., a metal or plastic).

As illustrated in FIG. 7B, the proximal anchor sections 160 can have a distal lip 166 and/or a proximal lip 168 that can engage with lip grooves 169 on the body 128 to help secure the proximal anchor 150 and prevent the proximal anchor 150 from sliding longitudinally along the body 128. In other embodiments, the proximal anchor 150 can be coupled to the body 28 in a variety of manners, such as, adhesives, cements, fasteners, threaded surfaces, interlocking surface structures and the like.

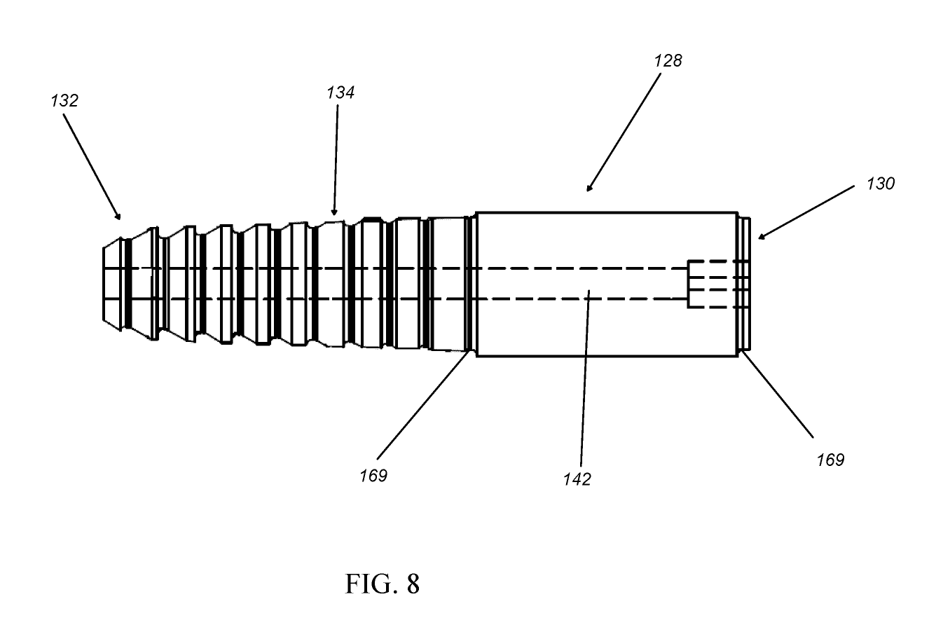

FIG. 8 illustrates an embodiment of the body 128 that is configured to couple with the proximal anchor sections 160. The body 128 can have lip grooves 169 towards the proximal end 130 that extend around the circumference of the body 128 and can accept the lips 166, 168 on the proximal anchor sections 160. The lip grooves 169 can help secure the proximal anchor 150 in the longitudinal direction. In the illustrated embodiment, the body 128 has a distal anchor 134 with grooves around the circumference of the body that help secure to the bone after inserting the body into the hole. The distal anchor 134 can also be tapered with the smallest diameter of the taper toward the distal end 132. This embodiment of the distal anchor 134 is configured to allow the body 128 to be pushed into the hole in the vertebrae without the need to rotate or screw in the body 128. In some embodiments, the body 128 can be cannulated forming a central lumen 142 to accommodate installation over a placement wire as is understood in the art. The cross section of the illustrated central lumen is circular but in other embodiments may be non-circular, e.g., hexagonal, to accommodate a corresponding male tool for installation or removal of the body 128 as explained below. In other embodiments, the body 128 may partially or wholly solid. In modified embodiments, the distal anchor 134 can include threads or can be formed without ridges or grooves.

In the illustrated embodiment embodiments, the body 128 and proximal anchor 150 can be made from allograft or substantially of allograft as described above. As discussed above, the allograft material advantageously promotes integration with the native bone structure. In some embodiments, it can be beneficial for the proximal anchor 150 not to integrate with the native bone structures, such as when the proximal anchor 150 needs to be removed later, or when bone growth around the proximal anchor 150 undesirably changes critical dimensions of the proximal anchor. In such embodiments, the proximal anchor sections 160 can be treated with a process to resist bone in-growth and integration.

In one embodiment of the device of FIGS. 7A and 7B, the body 128 has a length of about 25-30 millimeters and a diameter of about 5.5 millimeters. The proximal anchor 150 can have a length of about 12 millimeters and a diameter of about 10 millimeters.

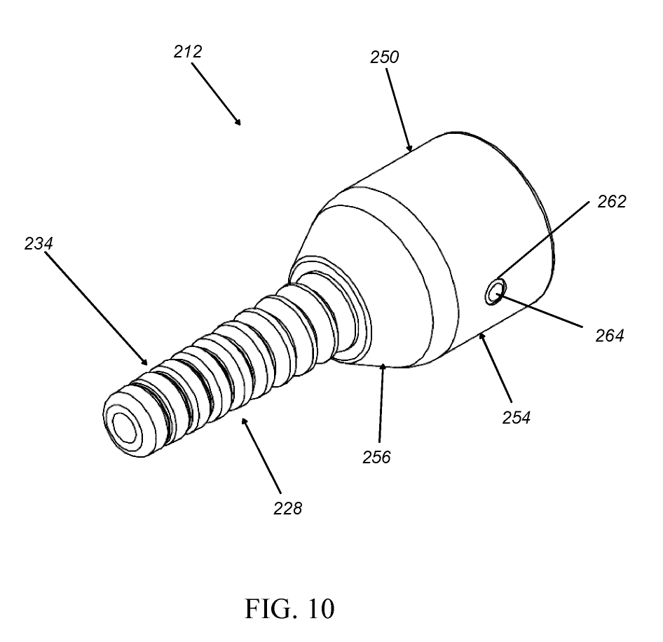

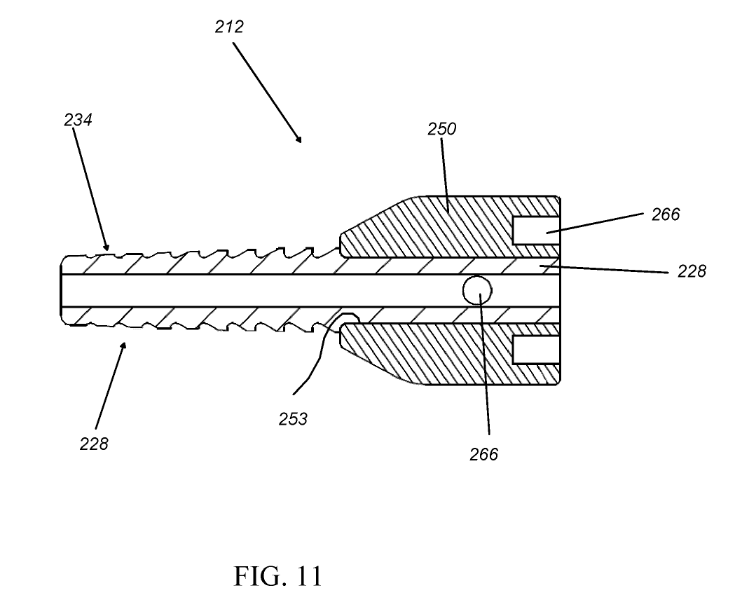

FIGS. 10 and 11 illustrate another embodiment of a device 212 having a proximal anchor 250 and a distal anchor 234. In the illustrated embodiment, the proximal anchor 250 has a cylindrical proximal portion 254 and a tapered distal portion 256. The cylindrical proximal portion 254 is configured to abut the inferior facet of the superior vertebrae and the tapered distal portion 256 is configured to fit in the countersinks 300 on the inferior vertebrae. Similar to described above for other embodiments, the proximal anchor 250 can be secured around the body 228 towards the proximal end.

In the embodiment illustrated in FIG. 10, the proximal anchor 250 can be secured to the body 228 with a pin or dowel 264. The dowel 264 can be inserted into a hole 262 in the proximal anchor 250 that extends from an exterior surface of the proximal anchor 250 through to the central lumen 256 of the proximal anchor 250. The body 228 can have a hole 266 that extends at least partially through the body 228. The dowel 264 can be inserted through the hole 262 in the proximal anchor 250 and into the hole 266 in the body 228. In some embodiments, the device 212 can be configured so that the dowel 264 can extend through one side of the proximal anchor 250, through the body 228, and at least partially through the other side of the proximal anchor 250. In some embodiment the dowel 264 can extend completely through the device 212 from one side of the proximal anchor 250, through the body 228, and through to the other side of the proximal anchor 250.

In some embodiments, the hole 262 can extend at an angle to the transverse plane that is normal to the longitudinal axis of the proximal anchor 250. The dowel 264 can have an interference fit in the holes 262 or can be secured through a variety of manners, such as, adhesives, cements, fasteners, threaded surfaces, interlocking surface structures and the like. For example, instead of a dowel, a fastener can be screwed into the proximal anchor 250 and into the body 228. In one arrangement, the pin or dowel 264 is also made of allograft. In a modified arrangement, the pin or dowel can be made of another material (e.g., a metal or plastic).

With reference to FIG. 11, in some embodiments the proximal anchor 250 can have a coupling feature 266 that can engage with an insertion tool for providing anti-rotational securement of the proximal anchor 250 during the implant procedure. Although illustrated as cylindrical cavities in FIG. 11, other embodiments of the coupling feature 266 may be contemplated by those skilled in the art in view of the disclosure herein.

In the illustrated embodiment embodiments, the body 228 and proximal anchor 250 can be made from allograft or substantially of allograft as describe above. As discussed above, the allograft material advantageously promotes integration with the native bone structure. In some embodiments, it can be beneficial for the proximal anchor 250 not to integrate with the native bone structures, such as when the proximal anchor 250 needs to be removed later, or when bone growth around the proximal anchor 250 undesirably changes critical dimensions of the proximal anchor. In such embodiments, the proximal anchor 250 can be treated with a process to resist bone in-growth and integration.

The embodiments described above in which the device comprises multiple pieces can be assembled and semi-permanently or permanently attached prior to implantation. In other embodiments, one or more of the assembly steps can occur at the surgical site. In other embodiments, the pieces can be pre-assembled and then permanently or semi-permanently attached at the surgical site.

Methods of implanting stabilization devices described above as part of a spinal stabilization procedure will now be described. Although certain aspects and features of the methods and instruments described herein can be utilized in an open surgical procedure, the disclosed methods and instruments are optimized in the context of a percutaneous or minimally invasive approach in which the procedure is done through one or more percutaneous small openings. Thus, the method steps which follow and those disclosed are intended for use in a trans-tissue approach. However, to simplify the illustrations, the soft tissue adjacent the treatment site have not been illustrated in the drawings.

In some embodiments of use, a patient with a spinal instability is identified. The patient is preferably positioned face down on an operating table, placing the spinal column into a normal or flexed position. A trocar optionally can then be inserted through a tissue tract and advanced towards a first vertebrae. In some embodiments, biopsy needle (e.g., Jamshidi™) device can be used. A guidewire can then be advanced through the trocar (or directly through the tissue, for example, in an open surgical procedure) and into the first vertebrae. The guide wire is preferably inserted into the pedicle of the vertebrae preferably through the pars (i.e. the region of the lamina between the superior and inferior articular processes). A suitable expandable access sheath or dilator can then be inserted over the guidewire and expanded to enlarge the tissue tract and provide an access lumen for performing the methods described below in a minimally invasive manner. In a modified embodiment, a suitable tissue expander (e.g., a balloon expanded catheter or a series of radially enlarged sheaths) can be inserted over the guidewire and expanded to enlarge the tissue tract. A surgical sheath can then be advanced over the expanded tissue expander. The tissue expander can then be removed such that the surgical sheath provides an enlarged access lumen. Any of a variety of expandable access sheaths or tissue expanders can be used, such as, for example, a balloon expanded catheter, a series of radially enlarged sheaths inserted over each other, and/or the dilation introducer described in U.S. patent application Ser. No. 11/038,784, filed Jan. 19, 2005 (Publication No. 2005/0256525), the entirety of which is hereby incorporated by reference herein.

A drill with a rotatable tip may be advanced over the guidewire and through the sheath. The drill may be used to drill an opening in the vertebrae. The opening may be configured for (i) for insertion of the body 28 of the bone stabilization device 12, (ii) tapping and/or (iii) providing a counter sink for the proximal anchor 50. In other embodiments, the step of drilling may be omitted. In such embodiments, the distal anchor 34 is preferably self-tapping and self drilling. In embodiments in which an opening is formed, a wire or other instrument can be inserted into the opening and used to measure the desired length of the body 28 of the device 12.

As will be explained below, the superior body structure (e.g., the superior vertebrae 10b) can be conformed to the device by providing a complementary surface or interface. In some embodiments, the superior vertebrae can be modified using a separate drill or reamer that is also used to from the countersink described above. In other embodiments, the drill that is used to form an opening in the inferior superior body can be provided with a countersink portion that is also used to modify the shape of the superior vertebrae 10b. In still other embodiments, the shape of the superior vertebrae 10b can be modified using files, burrs and other bone cutting or resurfacing devices to from a complementary surface or interface for the proximal anchor 50.

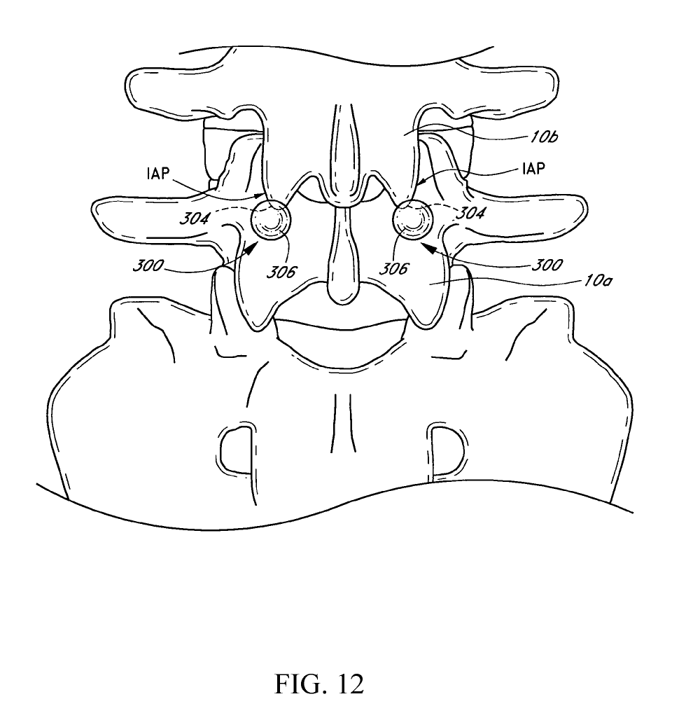

As mentioned above, a countersink can be provided for the proximal anchor 50. With reference to FIG. 12, a pair of counter sinks 300 are shown formed in or near the pars of the inferior vertebrae 10a. Each counter sink 300 is preferably configured to generally correspond to a distal facing portion 49a (see FIG. 4) of the proximal anchor 50. In this manner, the proximal anchor 50, in a final position, may be seated at least partially within the inferior vertebrae 10a. In the illustrated embodiment, the countersink 300 has a generally spherical configuration that corresponds generally to the spherical shape of the distal portion 49a of the proximal anchor 50 of the illustrated embodiment. In modified embodiments, the countersink 300 can have a modified shape (e.g., generally cylindrical, conical, rectangular, etc.) and/or generally configured to correspond to the distal portion of a proximal anchor 50 with a different shape than the proximal anchor illustrated in FIGS. 3-5.

The countersink 300 advantageously disperses the forces received by the proximal anchor 50 by the superior vertebrae 10b and transmits said forces to the inferior vertebrae 10a. The countersink 300 can be formed by a separate drilling instrument or by providing a counter sink portion on a surgical drill used to from an opening in the body 10b.

In addition or in the alterative to creating the countersink 300, the shape of the inferior articular process IAP (which can include the facet in some embodiments) of the superior vertebrae 10b can be modified in order to also disperse the forces generated by the proximal anchor 50 contacting, abutting and/or wedging against the superior vertebrae 10b. For example, as shown in FIG. 12, a portion 304 of the inferior articular process IAP of the superior vertebrae 10b that generally faces the proximal anchor 50 can be removed with the goal of dispersing and/or reducing the forces applied to the proximal anchor 50. In the illustrated embodiment, the inferior articular process is provided with a generally rounded recess 306 that corresponds generally to the rounded outer surface 49 of the proximal anchor 50. In modified embodiments, the inferior articular process IAP can be formed into other shapes in light of the general goal to reduce and/or disperse the forces applied to the proximal anchor 50. For example, in some embodiments, the inferior articular process IAP may be formed into a generally flat, blunt or curved shape. In other embodiments, the inferior articular process IAP may be configured to abut and/or wedge more efficiently with a proximal anchor 50 of a different shape (e.g., square, oval, etc.). In general, the countersink 300 and surface 306 provided for an increased contact surface between the superior vertebra 10b and the proximal anchor 50 and the inferior vertebra 10a and the proximal anchor 50. This contact area reduces stress risers in the device and the associated contact areas of the vertebrae. In addition, the windshield wiper affect is reduced as the forces transmitted to the proximal anchor 50 from the superior vertebrae are transmitted through the area formed by the countersink 300. Some embodiments of countersink and/or recess creating devices are disclosed in U.S. Patent Publication No. 11/296,881, filed on Dec. 8, 2005, which is hereby incorporated by reference herein.

In some embodiments, the clinician will have access to an array of devices 12, having, for example, different diameters, axial lengths, configurations and/or shapes. The clinician will assess the position of the body 28 with respect to the superior vertebrae and choose the device 12 from the array, which best fits the patient anatomy to achieve the desired clinical result. In some embodiments, the clinician will have access to an array of devices 12, having, for example, bodies 28 of different diameters, axial lengths. The clinician will also have an array of devices 12 with an array of proximal anchors 50, having, for example, different configurations and/or shapes. The clinician can choose the appropriate body 28 and proximal anchor 50 which best fits the patient anatomy to achieve the desired clinical result and then assess the position of the body 28 with respect to the superior vertebrae.

The body 28 of the fixation device can be advanced over the guidewire and through the sheath until it engages the vertebrae. The body 28 may be coupled to a suitable insertion tool prior to the step of engaging the fixation device 12 with the vertebrae. The insertion tool can be configured to engage the coupling 70 on the proximal end of the body 28 such that insertion tool may be used to rotate the body 28. In such an embodiment, the fixation device 12 is preferably configured such that it can also be advanced over the guidewire. Further disclosure of an insertion tool can be found in U.S. Patent Publication No. 11/296,881, filed on Dec. 8, 2005, which is hereby incorporated by reference herein.

The insertion tool can be used to rotate the body 28 thereby driving the distal anchor 34 to the desired depth within the pedicle of the vertebrae for those embodiments of stabilization device 12 having a distal anchor 34. The surgeon can stop rotating the body 28 before the distal end of the tool contacts the bone. In embodiments in which a countersink is formed, the tool can be rotated until the distal end sits within the countersink at which point further rotation of the tool will not cause the distal anchor to advance further as further advancement of the body 28 causes it to be released from the tool. In this manner, over advancement of the distal anchor 32 into the vertebrae can be prevented or limited.

The proximal anchor 50 can be integral with the body 28, or can be attached and/or coupled to the body 28 following placement (partially or fully) of the body 28 within the vertebrae. In some embodiments, the anchor 50 can be pre-attached and/or coupled to the body 28 prior to advancing the body 28 into the vertebrae.

In embodiments where the proximal anchor 50 is separate from the body 28, once the body 28 is in the desired location, the proximal anchor 50 is preferably advanced over the body 28 until it reaches its desired position. This can be accomplished by pushing on the proximal anchor 50 or by applying a distal force to the proximal anchor 50. In some embodiments, the proximal anchor 50 is pushed over the body 28 by tapping the device with a slap hammer or similar device that can be used over a guidewire. In this manner, the distal end of the device 12 is advantageously minimally disturbed, which prevents (or minimizes) the threads in the bore from being stripped.

The access site can be closed and dressed in accordance with conventional wound closure techniques and the steps described above may be repeated on the other side of the vertebrae for substantial bilateral symmetry as shown in FIGS. 1A and 1B. The bone stabilization devices 12 may be used alone or in combination with other surgical procedures such as laminectomy, discectomy, artificial disc replacement, and/or other applications for relieving pain and/or providing stability.

It should be appreciated that not all of the steps described above are critical to procedure. Accordingly, some of the described steps may be omitted or performed in an order different from that disclosed. Further, additional steps may be contemplated by those skilled in the art in view of the disclosure herein, without departing from the scope of the present invention.

As discussed above, in embodiments without a proximal anchor, the proximal end 30 of the device can limit motion by abutting and/or wedging against a surface of the superior vertebrae 10b as the superior vertebrae 10b moves relative to the inferior vertebrae 10a. In some embodiments, a mass or growth can form around the proximal end 30 of the stabilization device 12, which can provide a support against which the superior vertebrae 10b can abut. The mass can advantageously grow around the superior vertebrae 10a and provide an abutment that is contoured to the shape of the superior vertebrae 10a.

For embodiments having a proximal anchor 50, the proximal anchors 50 of the devices 12 extend above the pars such that they abut against the inferior facet of the superior adjacent vertebrae. In this manner, the proximal anchor 50 can form a wedge between the vertebra limiting compression and/or extension of the spine as the facet of the superior adjacent vertebrae abuts against the proximal anchor 50. In this manner, extension can be limited while other motion is not. For example, flexion, lateral movement and/or torsion between the superior and inferior vertebra is not limited or constrained. In this manner, the natural motion of the spine can be preserved, especially for those patients with mild or moderate disc conditions. Preferably, the devices are implantable through a minimally invasive procedure and, more preferably, through the use of small percutaneous openings as described above. In this manner, the high cost, lengthy in-patient hospital stays and the pain associated with open procedures can be avoided and/or reduced. In some embodiments, the devices 12 may be removed and/or proximal anchors 50 may be removed in a subsequent procedure if the patient's condition improves. Once implanted, it should be appreciated that, depending upon the clinical situation, the proximal anchor 50 may be positioned such that it contacts surfaces of the adjacent vertebrae all of the time, most of the time or only when movement between the adjacent vertebrae exceeds a limit.

In some instances, the practitioner may decide to use a more aggressive spinal fixation or fusion procedure after an initial period of using the stabilization device 12. In one particular embodiment, the bone stabilization device 12 or a portion thereof may be used as part of the spinal fixation or fusion procedure. In one such application, the proximal anchor 50 can be removed from the body 28. The body 28 can remain in the spine and used to support a portion of a spinal fixation device. For example, the body 28 may be used to support a fixation rod that is coupled to a device implanted in a superior or inferior vertebrae. Examples of such fusion systems can be found in U.S. patent application Ser. No 10/623,193, filed Jul. 18, 2003, the entirety of which is hereby incorporated by reference herein.

As mentioned above, in some embodiments described above, it may be advantageous to allow the proximal anchor 50 to rotate with respect to the body 28 thereby preventing the proximal anchor 50 from causing the distal anchor 34 from backing out of the pedicle. In some embodiments, engagement features (as described below) may be added to the proximal anchor 50 to prevent rotation of the proximal anchor 50.

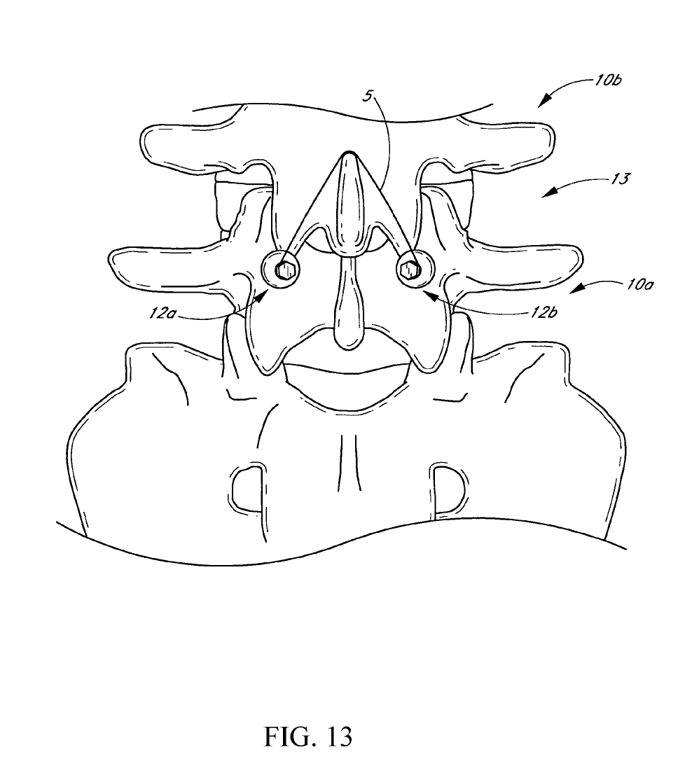

FIG. 13 illustrates a modified embodiment in which the first and second fixation devices 12a, 12b are coupled together by a member 5 that extends generally around or above the spinous process of the superior vertebra 10b. In this manner, the member 5 can be used to limit flexion of the spinal column. The member may comprise any of a variety of suitable structural members. In some embodiments, the member comprises a suture or wire that is tied to the proximal end of the bodies 28 or the proximal anchor. In some embodiments, various hooks or eyelets can be provided on the body or proximal anchor to facilitate coupling the member to the devices 12a, 12b.

The above described devices and techniques limit motion of the spine by providing an abutment or wedge surface on one vertebrae or body structure. The abutment surface contacts, abuts, and/or wedges against a portion of a second, adjacent vertebrae or body structure so as to limit at least one degree of motion between the two vertebra or body structure while permitting at least one other degree of motion. While the above described devices and techniques are generally preferred, certain features and aspects can be extended to modified embodiments for limiting motion between vertebra. These modified embodiments will now be described.

In the embodiments described above, the device is generally inserted into the spine from a posterior position such that a distal end of the device is inserted into the first, inferior vertebrae and a proximal end of the device contacts or wedges against the second, superior vertebrae. However, it is anticipated that certain features and aspects of the embodiments described herein can be applied to a procedure in which the device is inserted from a lateral or anterior site. In such an embodiment, the distal end or side portion of the device may contact or wedge against the second superior vertebrae. Such embodiments provide a contact or wedge surface which is supported by one body structure to limit of the motion of an adjacent body structure.

In the embodiments described above, it is generally advantageous that the proximal anchor be radiopaque or otherwise configured such that in can be seen with visual aids used during surgery. In this manner, the surgeon can more accurately position the proximal anchor with respect to the superior and inferior vertebra.

Preferably, the clinician will have access to an array of fixation devices, having, for example, different diameters, axial lengths and, if applicable, angular relationships. These may be packaged one or more per package in sterile or non-sterile envelopes or peelable pouches, or in dispensing cartridges which may each hold a plurality of devices. The clinician will assess the dimensions and load requirements, and select a fixation device from the array, which meets the desired specifications.

The components described herein may be sterilized by any of the well known sterilization techniques, depending on the type of material. Suitable sterilization techniques include, but not limited to heat sterilization, radiation sterilization, such as cobalt 60 irradiation or electron beams, ethylene oxide sterilization, and the like.

The specific dimensions of any of the of the described herein can be readily varied depending upon the intended application, as will be apparent to those of skill in the art in view of the disclosure herein. Moreover, although the present invention has been described in terms of certain preferred embodiments, other embodiments of the invention including variations in dimensions, configuration and materials will be apparent to those of skill in the art in view of the disclosure herein. In addition, all features discussed in connection with any some embodiments herein can be readily adapted for use in other embodiments herein. The use of different terms or reference numerals for similar features in different embodiments does not imply differences other than those which may be expressly set forth. Accordingly, the present invention is intended to be described solely by reference to the appended claims, and not limited to the preferred embodiments disclosed herein.

What is claimed is:

1. A spinal stabilization device, comprising:
an elongate body having a distal end and a proximal end, the distal end configured to be implanted in an inferior vertebrae and the proximal end configured to abut a superior vertebrae to limit at least one degree of movement between the superior vertebrae and the inferior vertebrae;
wherein the body is at least partially made of an allograft.

2. The spinal stabilization device of claim 1, wherein the allograft is cortical bone.

3. The spinal stabilization device of claim 1, wherein the entire body is made of allograft.

4. The spinal stabilization device of claim 1, further comprising a proximal anchor toward the proximal end of the body.

5. The spinal stabilization device of claim 4, wherein the proximal anchor is generally spherical with a lumen configured to couple to the body.

6. The spinal stabilization device of claim 4, wherein the proximal anchor comprises a generally cylindrical proximal portion and a tapered distal portion, and further comprises a lumen configured to couple to the body.

7. The spinal stabilization device of claim 4, wherein the proximal anchor is coupled to the body through an interference fit.

8. The spinal stabilization device of claim 4, wherein the proximal anchor is at least partially made of an allograft.

9. The spinal stabilization device of claim 4, wherein the proximal anchor comprises at least two sections that coupled together.

10. The spinal stabilization device of claim 9, wherein the sections of the proximal anchor are coupled together with dowels that extend at least partially through the sections.

11. The spinal stabilization device of claim 4, wherein the proximal anchor is secured to the body by a dowel.

12. The spinal stabilization device of claim 1, wherein the proximal end of the body is treated to resist bone in-growth.

13. The spinal stabilization device of claim 4, wherein the proximal anchor is treated to resist bone in-growth.

14. The spinal stabilization device of claim 1, further comprising a distal anchor on the distal end of the elongate body.

15. The spinal stabilization device of claim 14, wherein the distal anchor comprises a helical flange.

16. The spinal stabilization device of claim 14, wherein the distal anchor comprises a plurality of circumferential grooves.

17. The spinal stabilization device of claim 1, wherein the proximal end is configured for applying a torque to the body to rotate the body about a longitudinal axis of the body.

18. The spinal stabilization device of claim 17, wherein the proximal end has a cross-sectional area with a hexagonal shape.

19. A spinal stabilization system comprising:
a first stabilization device comprising an elongate body having a distal end and a proximal end, the distal end configured to be implanted in a left side of an inferior vertebrae and the proximal end configured to abut a left inferior articular process of a superior vertebrae to limit at least one degree of movement between the superior vertebrae and the inferior vertebrae, wherein the body is at least partially made of an allograft;
a second stabilization device comprising an elongate body having a distal end and a proximal end, the distal end configured to be implanted in a right side of an inferior vertebrae and the proximal end configured to abut a right inferior articular process of a superior vertebrae to limit at least one degree of movement between the superior vertebrae and the inferior vertebrae, wherein the body is at least partially made of an allograft; and
a member that connects the first stabilization device to the second stabilization device and extends generally around the spinous process of the superior vertebra to limit flexion of the spinal column.

20. The spinal stabilization system of claim 19, wherein the member is a wire.

21. A method of limiting extension between an inferior and superior body structure of a spine, the method comprising:
inserting a distal end of a stabilization device that is at least partially made from an allograft into the inferior body structure of the spine such that a proximal end of the stabilization device limits extension between the superior body structure and the inferior body structure.

22. The method of claim 21, wherein the entire stabilization device is made of an allograft.

23. The method of claim 21, further comprising the step of coupling a proximal anchor to the proximal end of the stabilization device.

24. The method of claim 23, wherein the proximal anchor is secured to the proximal end of the stabilization device through an interference fit.

25. The method of claim 23, wherein the proximal anchor is at least partially made of an allograft.
